(19) United States
(12) Patent Application Publication
(10) Pub. No.: US 2002/0177624 A1
(43) Pub. Date: Nov. 28, 2002

(54) Acetate-lactate buffering vaginal gel and for method of making same and treating bacterial vaginosis

(76) Inventors: Calvin Hanna, Little Rock, AR (US); Lindley A Cates, Conroe, TX (US)

Correspondence Address: JOE D. CALHOUN, 319 PRESIDENT CLINTON AVENUE, SUITE 205, LITTLE ROCK, AR, 72201, US

(21) Appl. No.: 09/809,835

(22) Filed: Mar. 16, 2001

(51) Int. Cl. A61K 31/19; A61K 33/04; A61K 33/42

(52) U.S. Cl. 514/557; 424/605; 424/709

(57) ABSTRACT

A composition of matter comprising a pH buffering system comprising a combination of lactic acid and related base salt that occur naturally in the human female body that, when applied to the surface of the vagina, maintains the pH level thereon at approximately the pH level of a healthy vagina. The composition my include a gel form, and also contain acetic acid as well as polyethylene glycol, glycerol and oxyquinoline.

### CROSS REFERENCES TO RELATED APPLICATIONS

Not applicable.

### FEDERALLY SPONSORED RESEARCH OR DEVELOPMENT

Not applicable.

### MICROFICHE APPENDIX

Not applicable.

### BACKGROUND OF THE INVENTION

(1) Field of the Invention.

The invention disclosed herein generally relates to a composition of matter formulated to provide a buffering system for maintaining the pH of the surface of the vagina at the appropriate level of acidity to assure the health thereof by assuring the continued presence of beneficial microorganisms rather than those facilitating or contributing to an unhealthy vaginal environment. The invention disclosed herein also generally relates to a method of making that composition of matter, as well as a method of using that composition of matter. Human females of all ages my use the composition disclosed herein. Besides being useful to females able to menstruate, it can also be useful to non-menstruating females such as pre-pubertal (for whom traditional estrogen supplementation would be ill advised) and menopausal females.

The composition disclosed herein is, in most general terms, a combination of acids and hydrogen-accepting substances (elements or molecules such as hydroxides or carbonates, or salts containing same) that occur naturally in the body. Said substances are combined with water (among other things) to produce a buffering system that, when applied to the surface of the vagina, maintains the pH level thereon at approximately the pH level of a healthy vagina.

The composition of matter disclosed herein facilitates the treatment of bacterial vaginosis using naturally occurring medicaments such as (for example) lactic acid (and possibly acetic or other acids) and/or conjugate salt thereof. More particularly, it relates to the field of a buffered acetate-lactate vaginal gel administered to compliment or replace the naturally occurring buffer used to maintain a vaginal pH of less that about 4.5 in the healthy vagina (such as, for example, a vagina manifesting a pH indicative of a proper amount of estrogen stimulation). By contrast, a vaginal environment not properly stimulated by estrogen maintains a pH near 7, and these females often develop bacterial vaginosis and vaginitis.

(2) Description of the Related Art Including Information Disclosed Under 37 C.F.R. 1.97 and 1.98.

Bacterial vaginosis is a condition occurring in or on the vagina of females of almost any age, commonly characterized by such symptoms as (a) vaginal wall fluid having a pH of greater than 4.5, (b) gray homogenous vaginal discharge, and/or (c) clue cells (epithelial cells studded with bacteria) on wet mount microscopic examination. With the precise cause(s) unknown, only the symptoms have heretofore been treated, rather prevention before the condition commences.

If left untreated, or if treatment exacerbates the symptoms, the female could manifest the condition known as bacterial vaginitis. Symptoms of bacterial vaginitis may include some of the same symptoms as bacterial vaginosis, but more pronounced and aggravated. The symptoms of bacterial vaginitis also include infection and/or lesions, with accompanying pus build-up.

Normal estrogen stimulated vaginal tissue produces excess glucose, that is stored by the epithelial cell lining as glycogen. These cells in turn leak glucose into the vaginal cavity where anaerobic bacteria at a highly acid pH converts the glucose to acetate and lactate (Boskey et al, Infection and Immunity, 67:5170-5175, 1999). One consequence is a buffering system comprising acetic and/or lactic acids, that essentially maintains the vaginal environment with an acidic pH of 4.5 or less. The resulting pH of 4.5 or less inhibits the growth of the non-lactate producing bacteria and other microorganisms (Eschenbach, D. A., Clin Obstet Gynecol, 26:186-202, 1983). As a result the vaginal microorganisms flourishing in the vaginal environment are predominantly non-pathogenic lactobacilli spp and related anaerobic bacteria. It is generally believed that vaginitis is precluded either by the presence of the lactobacilli and related anaerobic bacteria, or by their exclusion of other vaginosis-contributing microorganisms in the vagina.

The salts (such as, for example, those derived from lactic acid and acetic acid) produced along with their free acids form a strong natural buffer that maintains the acidic pH under the varying estrogen levels of the menstrual cycle. The lactate-lactic acid concentration is often about two percent by weight (2% w/w) on the healthy vaginal surface.

For unknown causes, the vagina may develop a watery discharge characterized by a pH greater than 4.5, with the presence of clue cells. Also, the fluid will liberate an amine (fishy) smell on warming. Together with other symptoms such as vulvar itching and irritation, the condition was classified by Gardner and Dukes as bacterial vaginosis (Gardner H. L., Dukes C. D., Science, 120:853,1954). The organism Haemophilus vaginalis was thought to be the causative agent and was renamed Gardnerella, but this has been shown to be in error (Spiegel, C. A. et al, J Clin Microbial, 18:170-7, 1983, New Eng J Med, 303:601-606, 1980) and the causative agents remain unknown.

Priestley, et al considers scented cleaning agents and soaps as causative in some cases (Priestley, C. I. F. et al, Genitourinary Med, 73:23-28, 1997). Vaginal douching, the use of oral contraceptives, and therapy with broad-spectrum antibiotics may lead to bacterial vaginosis.

Unlike bacterial vaginitis, bacterial vaginosis contains no areas of infection. The current treatment can involve oral metronidazole, which is often highly successful (Mengel, M. B. et al, Journal of Family Practice, 28:163-169 (1989). However, this treatment is not generally safe in females of child bearing age, primarily because of possible teratological effects on the developing embryo. Sometimes a woman does not initially know she is pregnant for a month or two while taking Metronidazole, and such accidental usage can nonetheless cause harm to a developing embryo or fetus. Since Metronidazole sometimes appears in mothers milk, the child is likely exposed to it upon feeding or possibly prior to that, in utero. The warning label for the use of Metrogel-Vaginal (3M-Pharmaceuticals) indicates such side effects as convulsive seizures, peripheral neuropathy, psychotic reactions and teratogenic effects.

The orally-administered antibiotic, clindamycin, is also recommended for treatment of bacterial vaginosis (Sweet, R. L., Am J Obstet Gynecol, 169: 479-82, 1993), and it is often highly successful. However, since clindamycin is non-natural, some people are allergic to it.

In general, the treatment regimen for bacterial vaginitis and vaginal yeast infections cannot be used in the treatment of bacterial vaginosis. One reason is that the treatment of bacterial vaginitis often includes the administration of broad-spectrum antibiotics, deadly to a wide variety of bacteria including the lactobacilli and related anaerobic bacteria that are believed to be partly responsible for maintaining a proper pH in the vaginal environment. There are a number of patents outlining the treatment of bacterial vaginitis and yeast infections. U.S. Pat. No.5,622,927 employed a mixture of folic acid, panthenol and/or allantoin, hydrolysate, lactose or dextrose, lactic acid, magnesium sulfate and sodium or ammonium chloride as suppositories, ointments, solutions or sprays to successfully treat vaginitis and vulvitis. In U. S. Pat. No. 5,667,817 an aqueous solution of chlorine dioxide was proposed for the therapy of microbiological infections of the vagina. Other patents outline the use of antifungal agents to eradicate yeast at the vaginal opening and vulva, U.S. Pat. Nos. 5,573,765 and 5,266,329.

Known is a product called Aci-Jel® sold by Ortho Pharmaceutical Corporation, for the treatment of vaginal infections (i.e., bacterial vaginitis); information about said product may be found at www.ortho-mcneil.com/products/info/aci-jel.htm. According to such product information, Aci-Jel® is a buffered acid jelly for intravaginal use, containing (among other ingredients) 0.9% glacial acetic acid, 0.025% oxyquinoline sulfate, 5% glycerol and potassium hydroxide. (The present invention also contains those quantities of substances.) That information on that product also claims that it acts to restore and maintain normal vaginal acidity through its buffer action. Although Aci-Jel® appears similar to the present invention at first blush, there are important distinguishing characteristics. One such distinction is the addition of lactic acid, a naturally occurring vaginal substance, to the buffering system of the present invention; the pH is determined by a combination of buffering characteristics of both lactate-lactic acid (pKₐ of 3.08) and another buffering agent such as acetate-acetic acid (pKₐ of 4.7). By contrast, the buffering characteristics of Aci-Jel® are determined solely by the acetate-acetic acid conjugate pair and, because the concentration is only 0.9% and the desired target pH. range is 3.8 to 4.2 (e.g., about 4.0), its buffering system is essentially using less acid to maintain a higher pH than the present invention. (The meager amount of 0.7% ricinoleic acid in Aci-Jel® virtually assures that this extremely weak acid does not function as a buffer.) At pH of 3.7, the present invention has about 1.1% combined free lactic and acetic acids (remaining for buffering); by contrast, Aci-Jel® has about 0.7% of free acetic acid (remaining for buffering) at pH 4.0. The 1.1% remaining for the present invention is more than Aci-Jel® had to begin with. In other words, the present invention has significantly greater buffering capacity.

Other distinguishing features between Aci-Jel® and the present invention are the lack of various Aci-Jel® ingredients that have detrimental effects. The present invention does not contain vegetable material such as tragacanth, acacia and egg albumin, which (absent preservatives) are likely subject to decomposition and/or mold growth; for this reason, the present invention (unlike Aci-Jel®) does not need to contain preservatives or anti-mold agents such as propylparaben. Nor does the present invention contain stannous chloride, thereby reducing the possibility of allergic reactions. Similarly, the present invention does not contain ricinoleic acid, the active ingredient in laxative caster oil, which can produce diarrhea.

### BRIEF SUMMARY OF THE INVENTION

Generally, the composition of matter disclosed here comprises a combination of acid and hydrogen-accepting substances, forming a buffering system essentially developing and/or maintaining the pH of the vaginal surface environment at about the same pH as a healthy vagina. More particularly, the combination comprises (or includes) acid(s) occurring naturally in the body, such as (for example) acetic acid, lactic acid, phosphoric acid and/or sulfuric acid, and combinations thereof; it also comprises naturally occurring hydrogen-accepting substance(s) such as potassium or sodium or calcium hydroxides or carbonates, and combinations thereof.

The pH of normal estrogen-stimulated vaginal fluid is between about 3.2 and 4.4. Where the desired pH and the pKa of the weak acid are the same, a very effective acid buffer system is created. (Henderson Hasselbalch Equation: pH=pKa+log [salt of weak acid / [weak acid].) The pKa of lactic acid is 3.08. By adding acetic acid with a pKa of about 4.7, a new optimal dual buffering system can result, to develop and maintain a pH in the range of between about 3.5 and 3.9, preferably around 3.7.

The composition of matter my also include formulations in the form of a gel, facilitating topical application to the vagina. For lactate-acetate vaginal gel to develop a pH of around 3.7, glycerol may be added to absorb water into the gel, to activate the buffer system. The combination may likewise include preservative formulations essentially providing the invention with stability of at least two years duration. One such version of the invention yields a stable, bland combination of lactic acid, acetic acid, glycerol, polyethylene glycol and oxyquinoline sulfate (preservative) buffered to a pH of around 3.7.

It is one primary object of the invention to provide a composition of matter capable of delivering a therapeutically effective amount of naturally occurring medicament(s) to the vaginal surface environment for the treatment of bacterial vaginosis.

Another primary object of the invention is to provide a composition of matter having a buffering system with significant great buffering capacity than current known systems.

It is another primary object of the invention to provide a combination capable of maintaining stability for at least 2 years.

Another object is to provide a method of making the aforementioned composition of matter. Another object is to provide a method of using the aforementioned composition of matter.

Other objects will become apparent to one having reasonable skill in this field, after reviewing this application.

### BRIEF DESCRIPTION OF THE SEVERAL VIEWS OF THE DRAWINGS

Not applicable.

### DETAILED DESCRIPTION OF THE INVENTION

The invention most generally provides a composition for maintaining the pH of a vagina at a healthy level, using acids and substances that occur naturally in the female body. The invention also includes a method of making said composition of matter, in addition to a method of using said composition.

For the sake of simplicity and to give the claims of this patent application the broadest interpretation and construction possible, the following definitions will apply:

1. The term “acetic acid” means acetic acid having the purity of or near that of glacial acetic acid.

2. The term “hydrogen-accepting” means accepting one or more hydrogen protons (H+).

3. The phrase “therapeutically effective amount” means the amount necessary to prevent the pH of the vaginal surface environment from varying beyond that of the healthy vaginal surface environment, properly stimulated by estrogen.

Also for the sake of simplicity, the conjunctive “and” in the written description may also be taken to include the disjunctive “or” in the written description, and vice versa, whenever necessary to give the claims of this patent application the broadest interpretation and construction possible. Likewise, when the plural form is used, it may be taken to include the singular form, and vice versa.

In its most general form, the invention includes a composition of matter comprising a pH buffering system comprising a combination of acid and hydrogen-accepting substance that occur naturally in the human female body that, when applied to the surface of the vagina, maintains the pH level thereon at approximately the pH level of a healthy vagina. Said pH is in the range of between about 3.2 and about 4.4. The present invention may develop and/or maintain the pH in the range of between about 3.5 and about 3.9; preferably said pH is in the range of about 3.7.

Said acid(s) may be selected from the group consisting of acetic acid, lactic acid, phosphoric acid and sulfuric acid, and combinations thereof. One of the important characteristics common to each of said members in said group, supporting the inclusion of each member in said group, is that each acid occurs naturally in the female body. Another common characteristic is each readily contributes to the formation of a buffering system, by temporarily donating hydrogen ion and accepting a cation to form a salt.

Said hydrogen-accepting substance may be selected from the group consisting of potassium hydroxide, sodium hydroxide, calcium hydroxide, potassium carbonate, sodium carbonate and calcium carbonate, and combinations thereof. One of the important characteristics common to each of said members in said group, supporting the inclusion of each member in said group, is that each substances is found naturally in the female body. Another common characteristic is that each readily contributes to the formation of a buffering system, by temporarily accepting hydrogen ion and donating an cation to form a salt. Such salts may be selected from the group consisting of acetate, lactate, phosphate and sulfate, in combination with said cation from said hydrogen-accepting substance.

One particular version of the invention comprises (or includes) a composition of matter wherein lactic acid is present in the range of between about 1.0% v/v and about 3.0% v/v. Preferably, said lactic acid is initially present in the range of about 2.0% v/v.

One particular version of the invention includes a composition of matter wherein another acid, acetic acid, is present in the range of between about 2.0% v/v and about 0.5% v/v. Preferably, said acetic acid is initially present in the range of about 0.9% v/v.

One particular version of the invention includes a composition of matter wherein said hydrogen-accepting substance is potassium hydroxide solution (2N) present in the range of between about 3.0% v/v and about 2.0% v/v. Preferably, said substance is potassium hydroxide solution initially present in the range of about 2.4% v/v.

Another aspect of the invention is a composition having gelatinous characteristics. The gel state results from the inclusion of polyethylene glycol. One particular version of the invention includes a composition of matter wherein said polyethylene glycol is polyethylene glycol 4500 present in the range of between about 75.0% v/v and about 50.0% v/v. Preferably said polyethylene glycol 4500 is initially present in the range of about 62.0% v/v. However, any similar gelation agent is acceptable, so long as it results in a composition of matter having a gel with characteristics suitable for transporting a therapeutically effective amount of buffering system material to the vaginal surface.

Another aspect of the invention is a composition of matter that includes a buffer activating component. One means of providing that characteristic is to include glycerol, which absorbs water or other fluid from the vaginal environment into the gel. It is believed that such fluid intake provides additional solvent, or a vehicle, to enhance the application of the composition of matter or to otherwise enhance its functioning. An acceptable range includes about 30% v/v to about 5.0% v/v; the preferable amount is about 15% v/v initially present in the final composition.

Another aspect of the invention is a composition having stability for at least two years. One means of accomplishing such a shelf life is to include oxyquinoline sulfate as a preservative. An acceptable range includes about 0.030% v/v to about 0.0020% v/v; the preferable amount is about 0.025% v/v initially present in the final composition.

A very specific version of the invention includes a composition of matter comprising a dual pH buffering system comprising a combination of lactic acid and hydrogen-accepting substance that occur naturally in the human female body that, when applied to the surface of the vagina, maintains the pH level thereon at approximately the pH level of a healthy vagina, wherein:

a. said lactic acid comprises lactic acid of about 2.0% v/v;

b. said substance comprises potassium hydroxide solution (2N) of about 2.4% v/v;

c. said polyethylene glycol comprises polyethylene glycol 4500 of about 62.0% v/v;

d. said glycerol comprises about 15.0% v/v;

e. said oxyquinoline sulfate comprises about 0.025% v/v; and

f. the remainder comprises water q.s. to 100%.

Another specific version of the invention further includes acetic acid of about 0.9% v/v.

A preferred formulation provides for a gel of medicaments comprising the buffering combination of lactate and acetate salts, and their counterpart lactic and acetic acids in a water solution. The starting material uses 2.0% (v/v) lactic acid and 0.9% (v/v) acetic acid, to which is added potassium hydroxide solution (2N) of 2.4% (v/v) to produce the lactate and acetate salts. A viscosity controlling agent such as polyethylene glycol may also be added, as may a fluid intake agent such as glycerol and a preservative such as 0.025% (v/v) of oxyquinoline sulfate.

Besides the aforementioned composition of matter, the invention disclosed herein also includes a method of making a composition of matter comprising a pH buffering system comprising a combination of lactic acid (plus possibly a second acid) and hydrogen-accepting substance that occur naturally in the human female body that, when applied to the surface of the vagina, maintains the pH level thereon at approximately the pH level of a healthy vagina. Said method includes the steps of mixing said lactic acid and substance in solution, then applying said solution to the vaginal surface

Said second acid and substance may be selected from the group consisting of acetic, lactic, phosphoric and sulfuric acid, potassium hydroxide, sodium hydroxide, calcium hydroxide, potassium carbonate, sodium carbonate and calcium carbonate, and combinations thereof (and salts thereof) as described hereinabove. In particular, said acid may be lactic acid in the range of about 2.0% v/v, and acetic acid in the range of about 0.9% v/v. Said substance may include potassium hydroxide solution (2N) of about 2.4% v/v. The method may also include mixing said acid and substance in polyethylene glycol 4500 in the range of about 62.0% v/v. The method may also include mixing said lactic acid, hydrogen-accepting substance and polyethylene glycol with glycerol in the range of about 15.0% v/v and oxyquinoline sulfate in the range of about 0.025% v/v.

A specific version of the method of making said composition of matter includes the steps of:

a. warming about 403 ml of polyethylene glycol 4500 to about 50° C. (ultimately to be about 62.0% v/v);

b. mixing therein about 97.5 grams of glycerol (ultimately to be about 15.0% v/v), about 13.0 grams of lactic acid (ultimately to be about 2.0% v/v), about 5.85 grams of acetic acid (ultimately to be about 0.9% v/v), about 16.0 grams of (2 N) potassium hydroxide solution (ultimately to be about 2.4% v/v), and about 50 grams of water (at this time about half of its ultimate q.s. of 100%), and stir at about 40° C. until completely mixed; and

C. cooling to about 21° C. and adding about 51 grams of water to bring to 100% q.s.

Moreover, a method of making said composition of matter may further include also mixing into the polyethylene glycol some oxyquinoline sulfate (ultimately to be about 0.025% v/v). To do this, mix 1.25 grams of oxyquinoline sulfate in 100 ml of water, then take about 13.0 grams of said aqueous solution and mix with the composition of matter of the invention.

Besides the aforementioned composition of matter and method of making same, the invention disclosed herein also includes a method of using a composition of matter comprising a pH buffering system comprising a combination of lactic acid (and possibly a second acid) and hydrogen-accepting substance that occur naturally in the human female body that, when applied to the surface of the vagina, maintains the pH level thereon at approximately the pH level of a healthy vagina. Said method may include the steps of contacting said composition with the vaginal surface. Said contacting may be accomplished by any means available. However, one preferred method is expelling said composition from a syringe-like applicator within the vaginal cavity.

Although application of the composition of matter may occur as often as is healthy, one preferred regime includes contacting occurring approximately twice daily in approximately regular intervals, for approximately seven consecutive days. Another application treatment regime further includes subsequent contacting approximately once daily, especially if bacterial vaginosis recurs.

A more specific account of using the gel includes a tamper resistant “star” seal at the opening of the 50 gram tube. Remove the cap and use the top of cap to twist off the seal. An applicator syringe (3.5 and 5.0 gram volume) is screwed to the top of the tube. Squeeze the tube to force the gel into the applicator. The full applicator is detached from the tube by turning the barrel. Insert the applicator into the vagina and press the plunger to deposit the vaginal gel. This application of the vaginal gel should be repeated twice daily for seven days. For repeated bacterial vaginosis the above application can be used once daily as prescribed.

The vaginal gel is delivered to the vagina with an applicator used once or twice per day. Clinical trials in cases of bacterial vaginosis have shown this natural therapy to be effective and safe with excellent compliance by the patients.

The following examples are included to illustrate the various formulations useful in this invention and the process for their preparations.

### EXAMPLE I

| Raw Materials | % v/v |
|---|---|
| Lactic acid, U.S.P. | 2.0 |
| Acetic acid, glacial, U.S.P. | 0.9 |
| Glycerol, U.S.P. | 15.0 |
| Polyethylene glycol 4500, U.S.P. | 62.0 |
| Oxyquinoline sulfate, U.S.P. | 0.025 |
| 2N Potassium hydroxide solution | 2.4 |
| Water, U.S.P. q.s. | 100% |

Place the polyethylene glycol, preferably 4500, in a suitable mixing container equipped with a mixer. Warm the ingredient to around 50° C. Add glycerol (and perhaps oxyquinoline sulfate), lactic acid, acetic acid and 2N Potassium hydroxide solution along with about one half of the q.s. water and stir at 40° C. until completely mixed. Cool to room temperature (about 21° C.) and bring to 100% (v/v) water. This medicament is placed in 50 gram tubes.

The solution formulated in Example I remained as an uncolored gel when stored at room temperature for two years. Stability studies on said gel revealed lactic acid at 2%±0.2% content, for two years.

### EXAMPLE II

| Raw Materials | % v/v |
|---|---|
| Lactic acid, U.S.P. | 2.0 |
| Glycerol, U.S.P. | 15.0 |
| Polyethylene glycol 4500, U.S.P. | 62.0 |
| Oxyquinoline sulfate, U.S.P. | 0.025 |
| 2N Potassium hydroxide solution | 2.4 |
| Water, U.S.P. q.s. | 100% |

### Results of Clinical Trial with “Example 1” to Test Bacterial Vaginosis

The clinical investigation of the vaginal gel according to Example 1 was carried out in the laboratory of Timothy J. Bell, D O, (PA), Fort Smith, AR using 18 patients. The criteria for bacterial vaginosis included at least three out of four of the following conditions: (a) gray homogenous discharge, (b) pH of the vaginal wall fluid 4.5 or greater, (c) positive clue cells on wet mount microscopic examination, (d) release of amine odor with addition of potassium hydroxide solution to the vaginal discharge fluid. In general there was additional vulva irritation associated with the diagnosis. Cure was the cessation of vaginal discharge for one month or longer as well as the disappearance of other symptoms. Four of the females met all four criteria for bacteria vaginosis out of the eighteen studied. Of the 18, seven had repeated symptoms after previous repeated treatment of metronidazole or clindamycin and one was refractory to all previous medications. Seventeen were cured including the refractory patient (vaginal pH 4) who also had less pelvic irritation. The one patient that remained uncured admittedly to continue the application treatment regime as directed.

### Statistics of the Eighteen Patients Tested

(a) age in years: eight, 21 to 27; eight, 31 to 38; one, 41; and one, 50;

(b) 3 used oral contraceptives of which 2 had repeated infections; 1 used vaginal douche;

(c) 2 complained that the gel of Example I was messy; and

(d) 1 had a repeated infection 8 months later which was cured with Example I.

The treatment of bacterial vaginosis with Example 1 was effective when used as directed. Many other patients using Example I did not return for follow up visits and were considered cured.

To date, about 5000 patients have been treated with Example I. A physician questionnaire has revealed that there have been eight cases of minor vaginal irritation and burning. The therapy consisted of using Example 1 twice a day for seven days also the patients with repeated bacterial vaginosis were continued once a night for thirty days.

### The pH Measurements with Example I Vaginal Gel

Measurement of pH of the pure gel of Example I is not reliable because the gel has insufficient water for the measurement. With the addition of water to the gel 1:03 (water) the pH was 4.66, on the addition of more water 1:06, pH 4.22, and 1:1 (water) to 3.72 and the same for 1:2 (water). As the gel is further diluted the pH remains around pH 3.7.

Those skilled in the art who have the benefit of this disclosure will appreciate that it may be used as the creative basis for designing devices or methods similar to those disclosed herein, or to design improvements to the invention disclosed herein; such new or improved creations should be recognized as dependant upon the invention disclosed herein, to the extent of such reliance upon this disclosure.

We claim:

1. A composition of matter comprising a pH buffering system comprising a combination of lactic acid and hydrogen-accepting substance that occur naturally in the human female body that, when applied to the surface of the vagina, maintains the pH level thereon at approximately the pH level of a healthy vagina.

2. A composition of matter described in claim 1 hereinabove, further comprising a second acid that occurs naturally in the human female body that, together with said hydrogen-accepting substance, also helps maintain said pH.

3. A composition of matter described in claim 1 hereinabove, wherein said pH is in the range of between about 3.5 and about 3.9.

4. A composition of matter described in claim 1 hereinabove, wherein said pH is in the range of about 3.7.

5. A composition of matter described in claim 2 hereinabove, wherein said second acid is selected from the group consisting of acetic acid, phosphoric acid and sulfuric acid, and combinations thereof.

6. A composition of matter described in claim 1 hereinabove, wherein said lactic acid is present in the range of between about 1.0% v/v and about 3.0% v/v.

7. A composition of matter described in claim 1 hereinabove, wherein said lactic acid is present in the range of about 2.0% v/v.

8. A composition of matter described in claim 2 hereinabove, wherein said acetic acid is present in the range of between about 2.0% v/v and about 0.5% v/v.

9. A composition of matter described in claim 2 hereinabove, wherein said acetic acid is present in the range of about 0.9% v/v.

10. A composition of matter described in claim 1 hereinabove, wherein said hydrogen-accepting substance is selected from the group consisting of potassium hydroxide, sodium hydroxide, calcium hydroxide, potassium carbonate, sodium carbonate and calcium carbonate, and combinations thereof.

11. A composition of matter described in claim 1 hereinabove, wherein said hydrogen-accepting substance is potassium hydroxide solution (2N) present in the range of between about 3.0% v/v and about 2.0% v/v.

12. A composition of matter described in claim 11 hereinabove, wherein said hydrogen-accepting substance is potassium hydroxide solution present in the range of about 2.4% v/v.

13. A composition of matter described in claim 1 hereinabove, further comprising polyethylene glycol.

14. A composition of matter described in claim 13 hereinabove, wherein said polyethylene glycol is polyethylene glycol 4500 present in the range of between about 75.0% v/v and about 50.0 % V/V.

15. A composition of matter described in claim 13 hereinabove, wherein said polyethylene glycol 4500 is present in the range of about 62.0% v/v.

16. A composition of matter described in claim 1 hereinabove, further comprising glycerol.

17. A composition of matter described in claim 16 hereinabove, wherein said glycerol is present in the range of between about 30.0% v/v to about 5.0% v/v.

18. A composition of matter described in claim 16 hereinabove, wherein said glycerol is present in the range of about 15% v/v.

19. A composition of matter described in claim 1 hereinabove, further comprising oxyquinoline sulfate.

20. A composition of matter described in claim 19 hereinabove, wherein said oxyquinoline sulfate is present in the range of between about 0.030% v/v to about 0.020% v/v.

21. A composition of matter described in claim 19 hereinabove, wherein said oxyquinoline sulfate is present in the range of about 0.025% v/v.

22. A composition of matter comprising a dual pH buffering system comprising a combination of lactic acid and hydrogen-accepting substance that occur naturally in the human female body that, when applied to the surface of the vagina, maintains the pH level thereon at approximately the pH level of a healthy vagina, wherein:
a. said lactic acid comprises lactic acid of about 2.0% v/v;
b. said substance comprises potassium hydroxide solution (2N) of about 2.4% v/v;
c. said polyethylene glycol comprises polyethylene glycol 4500 of about 62.0% v/v;
d. said glycerol comprises about 15.0% v/v;
e. said oxyquinoline sulfate comprises about 0.025% v/v; and
f. the remainder comprises water q.s. to 100%.

23. A composition of matter described in claim 22 hereinabove, further comprising acetic acid of about 0.9% v/v.

24. A method of making a composition of matter comprising a dual pH buffering system comprising a combination of lactic acid, a second acid and hydrogen-accepting substance that occur naturally in the human female body that, when applied to the surface of the vagina, maintains the pH level thereon at approximately the pH level of a healthy vagina, said method of making comprising the steps of mixing said acids and substance in solution, then applying said mixture to the vaginal surface.

25. A method of making described in claim 24 hereinabove, wherein said second acid and substance are selected from the group consisting of acetic acid, phosphoric acid, sulfuric acid, potassium hydroxide, sodium hydroxide, calcium hydroxide, potassium carbonate, sodium carbonate and calcium carbonate, and combinations thereof.

26. A method of making described in claim 24 hereinabove, wherein said lactic acid is lactic acid in the range of about 2.0% v/v.

27. A method of making described in claim 24 hereinabove, wherein said second acid is acetic acid in the range of about 0.9% v/v.

28. A method of making described in claim 24 hereinabove, wherein said hydrogen-accepting substance comprises potassium hydroxide solution (2N) in the range of about 2.4% v/v.

29. A method of making described in claim 24 hereinabove, further comprising mixing said acids and substance in polyethylene glycol 4500 in the range of about 62.0% v/v.

30. A method of making described in claim 24 hereinabove, further comprising mixing said acids, substance and polyethylene glycol with glycerol in the range of about 15.0% v/v and oxyquinoline sulfate in the range of about 0.025% v/v.

31. A method of making a composition of matter comprising a dual pH buffering system comprising a combination of lactic acid, a second acid and hydrogen-accepting substance that occur naturally in the human female body that, when applied to the surface of the vagina, maintains the pH level thereon at approximately the pH level of a healthy vagina, said method of making comprising the steps of:
a. warming about 403 grams of polyethylene glycol 4500 to about 50° C. (ultimately to be about 62.0% v/v);
b. mixing therein about 97.5 grams of glycerol, about 13.0 grams of lactic acid, about 5.85 grams of acetic acid, about 16.0 grams of potassium hydroxide solution, and about 50.0 grams of water, and stir at about 40° C. until fully mixed; and
c. cooling to about 21° C. and adding about 51 grams of water.

32. A method of making described in claim 31 hereinabove, further comprising also mixing into the polyethylene glycol about 13.0 grams of an aqueous solution of oxyquinoline sulfate obtained from mixing about 1.25 grams of oxyquinoline sulfate in 100 ml of water.

33. A method of using a composition of matter comprising a dual pH buffering system comprising a combination of lactic acid, a second acid and hydrogen-accepting substance that occur naturally in the human female body that, when applied to the surface of the vagina, maintains the pH level thereon at approximately the pH level of a healthy vagina, said method of using comprising the steps of contacting the vaginal surface with a therapeutically effective amount of said composition.

34. A method of using described in claim 33 hereinabove, wherein said contacting is accomplished by expelling said composition from a syringe-like applicator within the vaginal cavity.

35. A method of using described in claim 33 hereinabove, wherein said contacting occurs approximately twice daily in approximately regular intervals, for approximately seven consecutive days.

36. A method of using described in claim 35 hereinabove, further comprising subsequent contacting approximately once daily.
